(19) United States
(12) Patent Application Publication
(10) Pub. No.: US 2019/0015030 A1
(43) Pub. Date: Jan. 17, 2019

(54) SYSTEM, METHOD AND APPARATUS TO FACILITATE DRAWING BLOOD AND EXTRACTING OTHER BODILY FLUIDS

(71) Applicant: Michael James BARKER, Edgewater, MD (US)

(72) Inventor: Michael James BARKER, Edgewater, MD (US)

(21) Appl. No.: 15/647,293

(22) Filed: Jul. 12, 2017

(51) Int. Cl. A61B 5/15

(52) U.S. Cl. CPC A61B 5/150343; A61B 5/15115; A61B 5/150061; A61B 5/150412

(57) ABSTRACT

A system, method and apparatus are provided for facilitating the extraction of bodily fluids, including blood, in a medical setting. The disclosed apparatus is configured as a single unit integral apparatus composed of two opposing and interconnected flexible clip components. A proximal end of a unitary apparatus includes two opposing flexible clip components configured to flexibly yet firmly engage a thumb or finger of the medical professional undertaking the blood draw in a manner that secures the apparatus to the thumb or finger of the medical professional during the blood draw evolution. A distal end of the unitary apparatus includes two opposing flexible clip components configured to flexibly yet firmly engage a blood tube holder of a typical standard diameter to securely hold the blood tube holder in place in proximity to the thumb or finger of the medical professional during the blood draw evolution.

### BACKGROUND

### 1. Field of the Disclosed Embodiments

This disclosure is directed to a unique system, method and apparatus for facilitating the extraction of bodily fluids, including blood, in a medical setting.

### 2. Related Art

Diagnostic protocols have emerged over the course of the last several decades that require collection of various bodily fluids and particularly the drawing, and often remote testing, of blood. Typically, blood is drawn by a medical professional, including a phlebotomist, in a clinical setting. Individual blood draw tubes, which can be many in number for the reasons set forth below, are carefully handled, catalogued and often shipped off to a remote laboratory for particular testing for myriad purposes.

Very often, particular tests are run on particular blood samples which may be, for example, mixed with other substances in sterile vials at the time that the blood is drawn. It is not uncommon for a doctor to order a series of many and widely-varied tests of a particular patient requiring collection of a number of individual samples of blood, which may be, for example, shipped off to separate laboratories for particular testing.

It is not uncommon, therefore, when an individual patient arrives at a clinic to have her or his blood drawn to be confronted with a medical professional, often a phlebotomist, that is carrying a number of individual blood draw tubes that will be sequentially employed by the medical professional to draw several separate samples of the patient's blood to be separately tested for the specific purposes outlined by the doctor's orders.

Separately, it is becoming more and more common for a number of physiological reasons including, but not limited to, the proliferation of obesity, particularly in the United States, that a phlebotomist may choose to extract blood from a particular patient at one of the patient's extremities. Typically, such a blood draw deviates from the traditional blood draw in which a needle attached to a receptacle was inserted into a patient's arm and the multiple vials or tubes of blood were extracted using that conventional apparatus with which many are familiar. The advantage of the conventional method is that, based on the direct connection of the needle to the receptacle by which individual tubes of blood may be inserted for collecting of the blood, control of the entire evolution by the medical professional using two hands is fairly routine.

The newer protocol, which may include typically drawing blood from the back of a patient's hand, includes what may be considered a more cumbersome drawing device. Such a blood draw device 100 is shown generally in FIG. 1, and may be typically referred to as a butterfly needle blood draw set up, apparatus or system. This blood draw device 100 generally comprises a needle 110 that is typically mounted to some small (perhaps “winged”) mechanical apparatus 120 by which the individual undertaking the blood draw can control the needle 110 for precise insertion through the patient's skin into the patient's blood vessel in the back of the patient's hand to facilitate collecting of the blood. The general configuration of the mechanical apparatus 120 shown in FIG. 1 with protrusions 122,124 (or wings) extending substantially orthogonally to the needle leads to the reference to this configuration as the butterfly needle blood draw set up. The mechanical apparatus 120 and the needle 110 may be coupled to a tube 130 that generally leads to a separate receptacle or blood tube holder 140, that is conventionally configured to accommodate varying sizes of blood draw tubes into which the various blood draw tubes for a particular blood draw evolution can be sequentially inserted in the blood draw process.

The combination of the blood draw device 100 being comprised of multiple non-rigidly connected components with the requirement for multiple tubes of blood to be drawn in a single blood draw evolution combine to render the modern blood draw evolution incredibly cumbersome for even the most practiced phlebotomist.

When the medical professional or phlebotomist attempts to draw blood using a butterfly needle, the medical professional punctures the skin, advances the needle 110 into the vein, and then must transition to picking up the blood tube (or several blood tubes, in sequence) and inserting each of the blood tubes as it is to be used into the blood tube holder 140 to puncture the top of the blood tube to begin drawing blood.

The guiding of the blood tube into the blood tube holder 140 becomes increasingly difficult if the medical professional is attempting to stabilize the needle in the patient's arm with one hand, while using the other hand to reach for the blood tube and then guide the blood tube into the blood tube holder 140, which is conventionally hanging freely at the end of the tube 130 from the back end of the needle. Frequently, it takes multiple attempts to successfully grab the blood tube holder 140 and advance the blood tube with only one hand, i.e., the hand that is not stabilizing the needle 110 in the patient's vein. One simply may not have enough hands to carefully address patient comfort by, for example, attempting to ensure that the needle 110 in the mechanical apparatus 120 does not move or shift during an evolution that requires the engagement of a single blood draw tube in the blood tube holder 140 much less the swap-out of multiple blood draw tubes during the single blood draw evolution.

There are a number of conventional solutions which have been tried to address the above-indicated and similar problems. None of these “solutions” have proven successful in appropriately addressing the particular configuration problems outlined above in a manner that is simple enough to manufacture, and for individual users to employ to reasonably overcome the noted shortfalls in the cumbersome nature of the modern blood draw evolution. None of the proposed “solutions” has gained even limited acceptance generally because they do not specifically address the shortfalls in an easily employable manner.

### SUMMARY

Based on the foregoing, it would be advantageous to provide a device that may be specifically-sized and easily manufactured to address the noted shortfalls. Specifically, it may be advantageous to provide a device in which a blood tube holder may be secured to the phlebotomist's hand in a manner that is not cumbersome and supports the ability to more readily allow the medical professional to insert individual blood draw tubes into the blood tube holder secured to the phlebotomist's hand. Such a capability would increase the efficiency and safety of the blood draw evolution and provide additional patient comfort in that the phlebotomist would be able to maintain additional focus on ensuring that the needle apparatus did not move in the patient's vein during even a protracted blood draw evolution.

Exemplary embodiments of the systems, methods and devices according to this disclosure may provide a single unit integral apparatus that may each be comprised of two opposing flexible clip components.

In embodiments, a proximal end of a unitary apparatus may be comprised of one of the two opposing flexible clip components that may be configured to flexibly yet firmly engage a thumb or finger of the medical professional undertaking the blood draw in a manner that secures the apparatus to the thumb or finger of the medical professional during the blood draw evolution.

In embodiments, a distal end of the unitary apparatus may be comprised of another of the two opposing flexible clip components that may be configured to flexibly yet firmly engage a blood tube holder of any one of typical standard diameters to securely hold the blood tube holder in place in proximity to the thumb or finger of the medical professional during the blood draw evolution.

Exemplary embodiments may solve common problems in the blood draw field including, but not limited to, a general inability to effectively secure and/or control the blood tube holder of a butterfly needle blood draw setup, while obtaining blood specimens from a patient.

Exemplary embodiments may facilitate the ability of the phlebotomist to more easily guide the blood draw tube into the blood tube holder while generally maintaining concentration on attempting to stabilize the needle in the patient's vein.

Exemplary embodiments may effectively secure the blood tube holder in a manner that allows the phlebotomist to more easily employ her or his free hand to reach for the blood draw tube and then guide the blood draw tube into the blood tube holder, which is no longer simply hanging freely from the blood transport tube engaged to the back end of the needle.

Exemplary embodiments may increase efficiency in the blood draw evolution while ensuring patient safety and comfort by reducing the number of attempts required by the phlebotomist to successfully grab and secure the blood tube holder with the free hand in order to advance the blood draw tube into the blood tube holder.

In embodiments, the disclosed device may efficiently and more effectively secure the blood tube holder to the phlebotomist's thumb or finger, including over the top of the phlebotomist's glove, allowing freedom of one hand to maintain stabilization of the needle, while also securing the tube holder to the thumb or finger of that hand, in a readily available and accessible location in a comparatively secure manner.

Exemplary embodiments may be configured such that the opposing flexible clip components of the integral apparatus may be positioned substantially parallel to one another, substantially perpendicular to one another, or at any intermediate angle that a particular medical professional may prefer. Based on the ease of manufacture, an array of variable angles between the opposing flexible clip components of the integral apparatus may be provided to accommodate user preference.

In embodiments, the distal opposing flexible clip component that is configured to accommodate the blood tube holder may additionally include protrusions or wings along the sides that are substantially configured to restrict the blood tube holder from advancing too far particularly during evolutions in which multiple blood draw tubes, are inserted and extracted from the blood tube holder, thereby maintaining a position of the blood tube holder substantially centered over the thumb or the finger to which the proximal opposing flexible clip component of the integral apparatus may be secured.

In embodiments, the integral apparatus may be formed of nonporous materials to reduce the potential for contaminant transmission leading to infection of a patient.

In embodiments, the integral apparatus may be formed of, or coated with, antimicrobial substances in order to reduce the potential for contaminant transmission leading to infection of a patient.

In embodiments, the integral apparatus may be simply formed and configured of disposable materials in order that each individual integral apparatus maybe employed only for a single use to reduce any potential for contaminant transmission between patients.

These and other features, and advantages, of the disclosed systems and methods are described in, or apparent from, the following detailed description of various exemplary embodiments.

### BRIEF DESCRIPTION OF THE DRAWINGS

Various exemplary embodiments of the disclosed unique system, method and apparatus for facilitating the extraction of bodily fluids, including blood, in a medical setting, will be described, in detail, with reference to the following drawings, in which:

FIG. 1 illustrates a schematic diagram of a conventional butterfly needle blood draw setup that may be usable with the system, method and/or apparatus according to this disclosure;

FIGS. 2A and 2B illustrate separate schematic diagrams of a first exemplary embodiment of integral blood tube holder apparatus according to this disclosure;

FIGS. 3A and 3B illustrate separate schematic diagrams of a second exemplary embodiment of integral blood tube holder apparatus according to this disclosure;

FIG. 4 illustrates a schematic diagram the first exemplary embodiment of the integral blood tube holder apparatus according to this disclosure in use secured to a thumb of a user; and

FIG. 5 illustrates a flowchart of an exemplary method for employing an integral blood tube apparatus according to this disclosure in a blood draw evolution.

### DETAILED DESCRIPTION OF EMBODIMENTS

The disclosed unique system, method and apparatus for facilitating the extraction of bodily fluids, including blood, in a medical setting, will be described as being particularly usable for that purpose. This single real-world application for the system, method and apparatus according to this disclosure should, however, not be considered as limiting the disclosed embodiments to only that purpose. Rather, the disclosed embodiments are intended to provide an overview of a particular real-world scenario for employment of such a system, method and/or apparatus. Any advantageous employment of a similarly configured device that may be used to supplement the capacity of a user to secure a tube-like device to one hand in order to free the other hand to engage components with that tube-like device that may benefit from the teachings of the disclosed embodiments are intended to be encompassed by the detailed description that follows.

Reference will be made to a substantially integral device that may be molded, or otherwise formed, of materials that will result in a substantially rigid structure with limitedly compliant openings at each end according to the described schemes. The disclosed schemes may include techniques for forming such an integral device from varying material compositions that may result in a substantially nonporous structure, which may optionally include some antimicrobial substances to reduce the potential for contaminant transmission in medical employment scenarios. Exemplary embodiments described and depicted in this disclosure should not be interpreted as being specifically limited to any particularly limiting material composition for the composition of the device, or to any particular method for forming the device as an integral structure, or as a structure of multiple connected components. Further, the exemplary embodiments described and depicted in this disclosure should not be interpreted as specifically limiting the configuration of any of the component parts, or to any limiting dimensions as may be implied by the detailed description that follows. All references to particular configurations and/or employment scenarios are intended to be illustrative only and are not intended to limit the disclosed concepts, compositions, processes, techniques, methods, systems and devices in any manner. It should be recognized that any advantageous use of the disclosed structures and schemes for providing a capacity to anchor a particular device, including a tube-like structure, to one or more of the digits of a user's hand that may advantageously employ systems, methods, techniques, and processes such as those discussed in detail in this disclosure is contemplated as being included within the scope of the disclosed exemplary embodiments.

In this regard, the disclosed systems, methods, apparatus and/or devices will be described as being particularly adaptable to the specific employment scenario of drawing blood from a patient as described in the background section of this disclosure. Again here, it must be noted that this description should not be considered limiting as to any intended use of a physical structure similar to that disclosed herein.

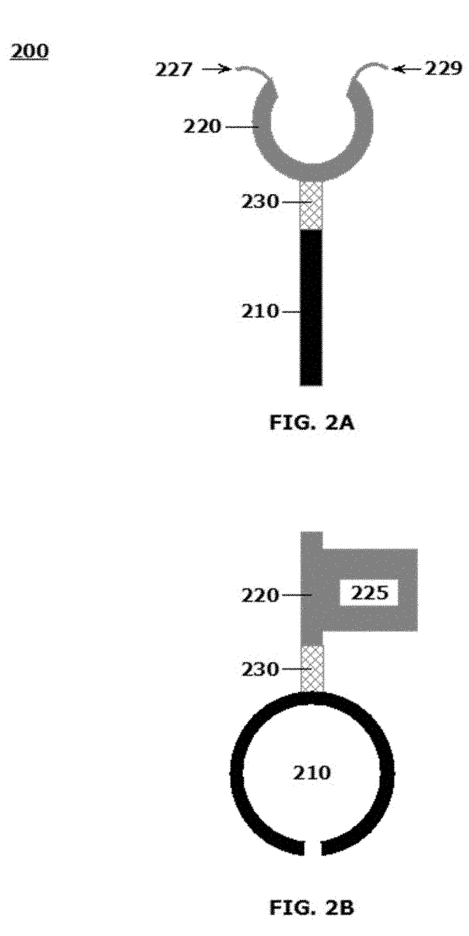

FIGS. 2A and 2B illustrate separate schematic diagrams of a first exemplary embodiment of integral blood tube holder apparatus 200 according to this disclosure. As shown in FIGS. 2A, and 2B, the integral blood tube holder apparatus 200 may include a lower portion 210, an upper portion 220 and a connecting portion 230. In the first exemplary embodiment shown in FIGS. 2A and 2B, a plane of the upper portion 220 may be set at substantially right angles to a plane of the lower portion 210. The connecting portion 230 may be in a form of an axially protruding member that connects the upper portion 220 at some interval distance from the lower portion 210. In embodiments, the interval distance may be reduced to almost zero. In yet other embodiments, the connecting portion 230 may be removed completely. In such embodiments, the upper portion 220 may be formed directly in contact with the lower portion 210.

The lower portion 210 may be configured to securely engage at least one of a thumb or finger of the user. The lower portion 210 may be configured to accommodate the thumb or finger of the user in a medical glove. The lower portion 210 may be configured as a flexible ring with an opening at its lower apex such that the two sides of the flexible ring generally constitute to opposing flexible legs. The lower portion 210 may be generally in a configuration of a toy plastic ring. The lower portion 210 may be formed integrally with the balance of the integral blood tube holder apparatus 200, or may be formed as a separate component. The lower portion 210 may be formed of a plastic, a polymer, a metal, or other generally flexible material that is resilient enough to open around the phlebotomist's thumb in a manner that causes the individual legs to securely engage the phlebotomist's thumb.

The upper portion 220 may be configured to securely engage a blood tube holder or other typically cylindrical component, which may be positioned in an opening formed by a wall of the upper portion 220. The upper portion 220 may optionally include protrusions 227, 229 generally in the plane of the upper portion 220. Such protrusions 227, 229 may facilitate expanding the opening formed by the wall of the upper portion 220 in order to mechanically spread the opposing resilient legs of the upper portion 220 so as to accommodate the insertion of the blood tube holder therebetween. Separately, such protrusions 227, 229 may form attach points for a closure mechanism over the gap formed in the top of the upper portion 220 in order to additionally mechanically secure the blood tube holder in the upper portion 220. The blood tube holder or other typically cylindrical component inserted between the legs of the upper portion 220 may be held in position by the resilient nature of the opposing legs of the upper portion 220. The upper 220 may be configured to accommodate the blood tube holders or other typically cylindrical components of varying diameters. In embodiments, the upper portion 220 may come in varying sizes as integrally formed with the lower portion 210, or as separately formed and individually attachable to the lower portion 210, directly or via a connecting portion 230. The upper portion 220 may be formed of a same or a different material than the lower portion 210. The upper portion 220 may itself be formed of a plastic, a polymer, a metal, or other generally flexible material that is resilient enough to open around the blood tube holder or other cylindrical component in a manner that causes the individual legs to securely engage the blood tube or other cylindrical component.

The upper portion 220 may include extensions 225, which may be in the form of “wings,” and which may be provided to contact a protrusion on the blood tube holder or other cylindrical component in order to restrict the movement of the blood tube holder or other cylindrical component in a direction orthogonal to the plane of the upper portion 220. In embodiments, these extensions 225 may restrict movement of the blood tube holder from advancing too far such that a position of the blood tube holder may be maintained substantially centered over the lower portion 210, and thereby centered in use over the user's thumb or finger to which the lower portion 210 is engaged.

Certain nonlimiting examples of dimensions of a particular exemplary embodiment of the configuration shown in FIGS. 2A and 2B may include the following. A cross-section of each of the individual lower portion 210, upper portion 220 and connecting portion 230 (where present) may be in a range of approximately 5 mm. A cross-sectional profile of each of the legs of the lower portion 210 and the upper portion 220, and cross-sectional profile of the connecting portion 230, may be in any known geometric configuration including round, square, rectangular, triangular, or other typical geometric configuration, and combinations thereof. A diameter of the opening in the upper portion 220 may be in a range of, for example, 22 mm. The extensions 225 of the upper portion 220 may extend, for example, as much as 15 mm or more in a direction orthogonal to the plane of the upper portion 220, and may separately extend vertically in a range of 10 mm or greater in order to particularly accommodate a size of a blood tube holder. As indicated above, these representative dimensions are intended to be illustrative and non-limiting to a particular size of a configuration of any of the individual components or component elements comprising the integral blood tube holder apparatus 200 shown in FIGS. 2A and 2B.

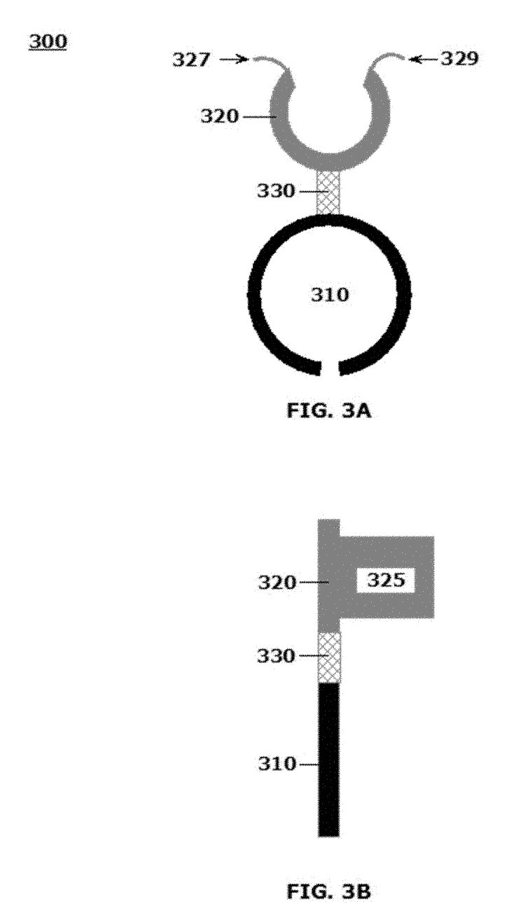

FIGS. 3A and 3B illustrate separate schematic diagrams of a second exemplary embodiment of integral blood tube holder apparatus 300 according to this disclosure. Like components between the configuration shown in FIGS. 2A and 2B and that shown in FIGS. 3A and 3B are similarly numbered for ease of explanation.

The integral blood tube holder apparatus 300 may include a lower portion 310, an upper portion 320 and a connecting portion 330.

A principal difference between the second exemplary embodiment shown in FIGS. 3A and 3B and the first exemplary embodiment shown in FIGS. 2A and 2B, is that the plane of the upper portion 320 and of the lower portion 310 are substantially the same. It should be recognized that, in embodiments, whether integrally formed, or formed to be connected to one another, the blood tube holder apparatus such as that shown in FIGS. 2A, 2B, 3A and 3B may be formed such that the planes of the respective upper portions 220, 320 may be rotated or positioned to be at virtually any angle with respect to the lower portions 210, 310. Such angle may vary between zero and 90° and may be preset in the manufacturing process by which an integral blood tube holder apparatus 200, 300 may be formed, or may be “settable” by a user in embodiments in which the upper portions 220, 320 are formed separately from and configured to be connected to the lower portions 210, 310.

The connecting portion 330 in this second exemplary embodiment may be similarly in a form of an axially protruding member that connects the upper portion 320 at some interval distance from the lower portion 310. As in the configuration shown in FIGS. 2 A and 2B, in embodiments, the interval distance between the upper portion 320 in the lower portion 310 may be reduced to almost zero. In yet other embodiments, the connecting portion 330 may be removed completely. In such embodiments, the upper portion 320 may be formed directly in contact with, or be formed to be connected directly to, the lower portion 310.

The lower portion 310 may be similarly configured to securely engage at least one of a thumb or finger of the user in the manner discussed above with respect to the lower portion 210 shown in FIGS. 2A and 2B. Again here, the lower portion 310 may be configured to accommodate the thumb or finger of the user in a medical glove.

The upper portion 320 may also be similarly configured to securely engage a blood tube holder or other typically cylindrical component, which may be positioned in an opening formed by a wall of the upper portion 320 substantially in the manner described in detail above with regard to the upper portion 220 shown in FIGS. 2A and 2B. Here too, the upper portion 320 may optionally include protrusions 327, 329 generally in the plane of the upper portion 320. Such protrusions 327, 329 may again facilitate expanding the opening formed by the wall of the upper portion 320 in order to mechanically spread the opposing resilient legs of the upper portion 320 so as to accommodate the insertion of the blood tube holder therebetween.

The upper portion 320 may similarly include extensions 325, which may be in the form of “wings,” and which may be provided to contact a protrusion on the blood tube holder or other cylindrical component in order to restrict the movement of the blood tube holder or other cylindrical component in a direction orthogonal to the plane of the upper portion 320 in the manner described above with respect to the extensions 225 shown in FIGS. 2A and 2B.

The certain nonlimiting examples of the dimensions of a particular exemplary embodiment of the configuration shown in FIGS. 2A and 2B may be equally applicable to the configuration of the integral blood tube holder apparatus 300 shown in FIGS. 3A and 3B.

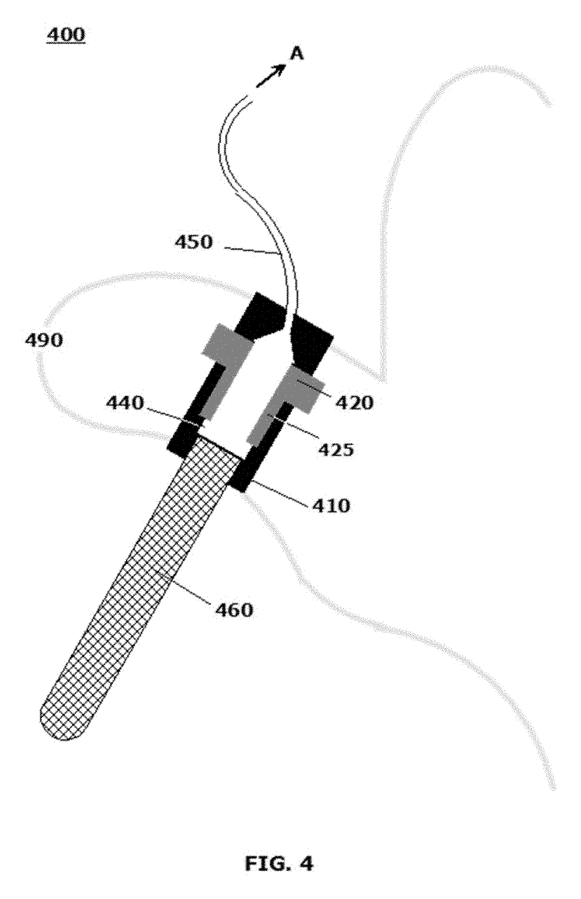

FIG. 4 illustrates a schematic diagram 400 of the first exemplary embodiment of the integral blood tube holder apparatus according to this disclosure, in use, secured to a thumb of a user (shown in profile as element 490).

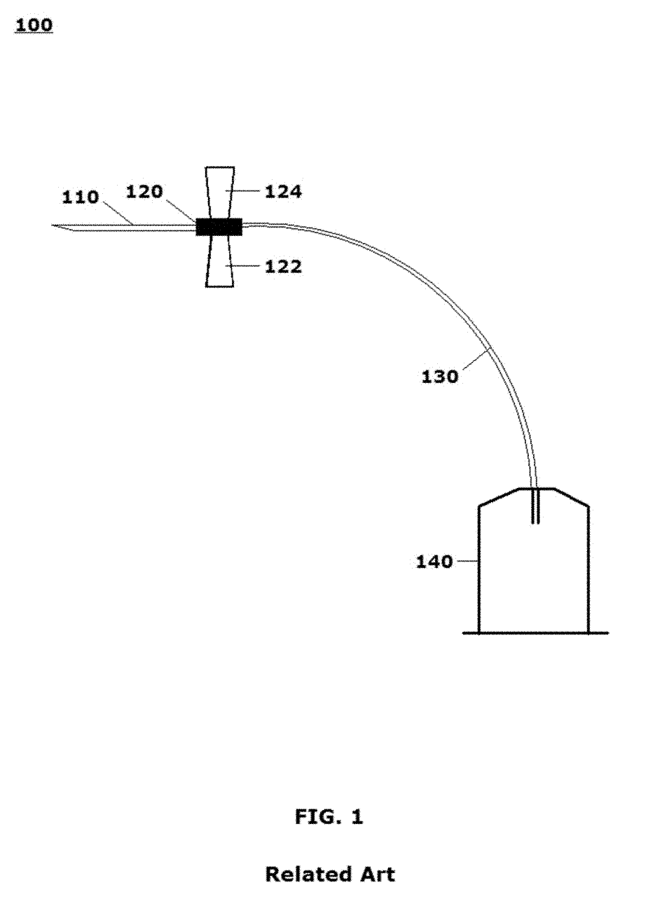

As shown in FIG. 4, a lower portion 410 is engaged to surround the user's thumb 490, and to preferably surround a medical glove worn by the user over her or his thumb 490. As depicted, a plane of the upper portion 420 is orthogonal to a plane of the lower portion 410. In this view, an opening in the top of the upper portion 420 with its extensions 425 accommodates insertion of a blood tube holder 440, which is inserted into, and engaged by, the upper portion 420. As shown, the blood tube holder 440 is in fluid communication with a tube 450 that extends from the blood tube holder 440 in a direction A, and opposite the end of the tube 450 being connectable to a needle or needle holder apparatus in the manner substantially shown in FIG. 1.

In the configuration shown in FIG. 4, the user's right hand, as depicted, may be used to maintain secure positioning of the needle in the patient's vein while the presence of the integral blood tube holder apparatus provides an anchor point for accommodating the blood tube holder 440 such that one or more blood draw tubes 460 may be insertable into the securely positioned blood tube holder 440 during a blood draw evolution undertaken in the manner described above.

As indicated, the blood tube holder apparatus according to this disclosure may be formed of substantially any nonporous material provided that individual opposing legs of the upper portion and of the lower portion of the formed structure may resiliently adapt themselves to securely accommodate a thumb or finger of the user in a lower portion and a blood tube holder or other similar cylindrical component in an upper portion. The nonporous nature of the formed structure is intended to limit transmission of contaminants in a vicinity of open wounds on a particular patient to thus avoid incidents of infection. Separately, the material from which the blood tube holder apparatus may be formed may include an antimicrobial agent such that potentially infecting contaminants may be mitigated in their contact with the structure of the blood tube holder apparatus.

The simplicity of the design and construction of the disclosed blood tube holder apparatus is intended, among other objectives, to provide a low-cost, easily manufactured apparatus for securing a blood tube holder during a blood draw evolution. The low-cost nature of the device is intended to promote disposal after a single use in order to further reduce the potential for cross-contamination between patients.

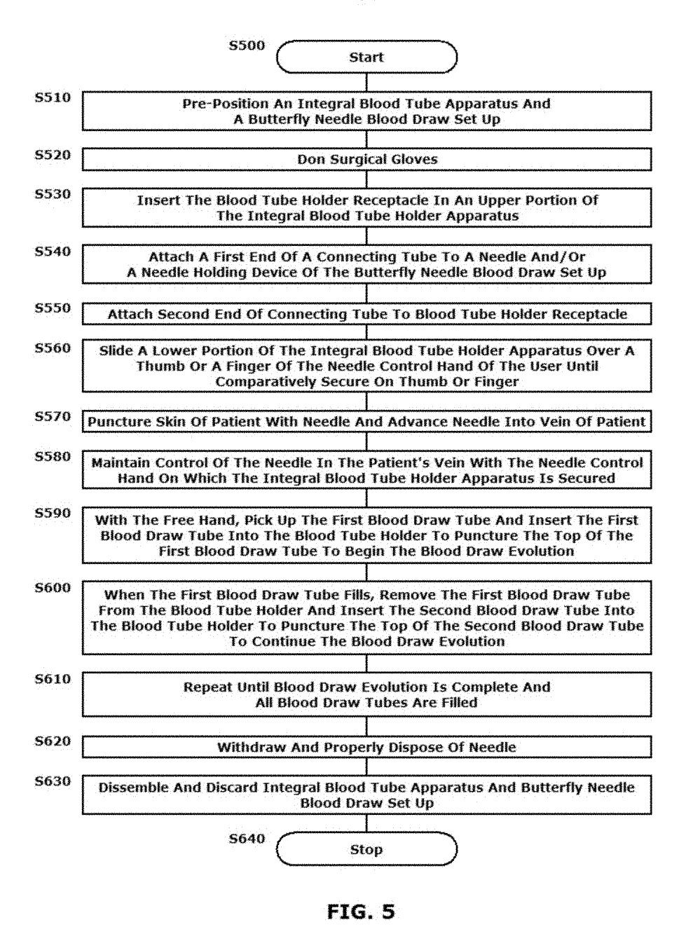

The disclosed embodiments may include a method for employing an integral blood tube apparatus according to this disclosure in a blood draw evolution. FIG. 5 illustrates a flowchart of such an exemplary method. As shown in FIG. 5, operation of the method commences at Step S500 and proceeds to Step S510.

In Step S510, an integral blood tube apparatus according to this disclosure and a butterfly needle blood draw set up, or similar blood draw device, may be pre-positioned. Operation of the method proceeds to Step S520.

In Step S520, a user may don medical gloves. Operation of the method proceeds to Step S530.

In Step S530, a blood tube holder receptacle of the butterfly needle blood draw set up may be inserted in an upper portion of the integral blood tube holder apparatus substantially in the manner described above. Operation of the method proceeds to Step S540.

In Step S540, a first end of a connecting tube may be attached to a needle and/or to a needle holding device, as appropriate. Operation of the method proceeds to Step S550.

In Step S550, a second end of the connecting tube may be attached to the blood tube holder receptacle secured within the integral blood tube holder apparatus. Operation of the method proceeds to Step S560.

In Step S560, a lower portion of the integral blood tube holder apparatus may be slid over a thumb or finger of a needle control and of the user until the integral blood tube holder apparatus is securely affixed to the thumb or finger. Operation of the method proceeds to Step S570.

In Step S570, the user may undertake a typical blood draw evolution by puncturing the skin of the patient with the needle and advancing the needle into the vein of the patient. Operation of the method proceeds to Step S580.

In Step S580, control of the needle may be maintained in the patient's vein with the needle control and of the user on which the integral blood tube holder apparatus may be secured to a thumb or a finger of the use. Operation of the method proceeds to Step S590.

In Step S590, the user may pick up the first blood draw tube with her or his free hand, and insert the first blood draw tube into the blood tube holder to puncture the top of the first blood draw tube to begin the blood draw evolution. Operation of the method proceeds to Step S600.

In Step S600, when the first blood draw tube is full, the first blood draw tube may be removed from the blood tube holder and a second blood draw tube may be inserted into the blood tube holder to puncture the top of the second blood draw tube to continue the blood draw evolution. Operation of the method proceeds to Step S610.

In Step S610, the above steps may be repeated until the blood draw evolution is complete and all blood draw tubes are filled. Operation of the method proceeds to Step S620.

In Step S620, when the blood draw evolution is complete, the needle may be withdrawn and properly disposed of. Operation of the method proceeds to Step S630.

In Step S630, the integral blood draw apparatus and the butterfly needle blood draw set up may be dissembled and discarded appropriately. Operation of the method proceeds to Step S640, where operation of the method ceases.

The above-described exemplary systems, methods, apparatus, and/or devices reference certain conventional components, materials, and real-world use cases to provide a brief, general description of suitable operating and integration environments in which the subject matter of this disclosure may be implemented for familiarity and ease of understanding.

Those skilled in the medical and phlebotomy fields of endeavor will appreciate that other embodiments of the disclosed subject matter may be practiced in many disparate blood draw and similar bodily fluid collection systems, techniques, processes and/or devices, including various structural components for providing secure attachment of a conventional blood draw apparatus to a user's thumb or finger in a manner that promotes security of the blood tube holder to facilitate the blood draw evolution.

The exemplary depicted sequence of method steps represents one example of a corresponding sequence of acts for implementing the functions described in the steps of the above-outlined exemplary method. The exemplary depicted steps may be executed in any reasonable order to carry into effect the objectives of the disclosed embodiments. No particular order to the disclosed steps of the method is necessarily implied by the depiction in FIG. 5, except where a particular method step is reasonably a necessary precondition to execution of any other method step.

Although the above description may contain specific details, they should not be construed as limiting the claims in any way. Other configurations of the described embodiments of the disclosed systems, methods, apparatus, devices, schemes and/or techniques are part of the scope of this disclosure.

It will be appreciated that various of the above-disclosed and other features and functions, or alternatives thereof, may be desirably combined into many other different systems or applications. Also, various alternatives, modifications, variations or improvements therein may be subsequently made by those skilled in the art which are also intended to be encompassed by the following claims.

We claim:

1. A device to facilitate blood draw, comprising:
a first substantially circular engaging portion that is configured to engage at least one of a thumb or a finger of a user; and
a second substantially circular engaging portion that is configured to engage at least one of a blood tube holder or another substantially tubular shaped component,
the first substantially circular engaging portion and the second substantially circular engaging portion being mechanically coupled to each other.

2. The device of claim 1, the first substantially circular engaging portion defining a first plane that is orthogonal to an axis at a center of the first substantially circular engaging portion, and the second substantially circular engaging portion defining a second plane that is orthogonal to an axis at a center of the second substantially circular engaging portion.

3. The device of claim 2, the first substantially circular engaging portion and the second substantially circular engaging portion being mechanically coupled to each other in a configuration in which the first plane and the second plane are perpendicular to one another.

4. The device of claim 2, the first substantially circular engaging portion and the second substantially circular engaging portion being mechanically coupled to each other in a configuration in which the first plane and the second plane define a single plane.

5. The device of claim 2, the first substantially circular engaging portion and the second substantially circular engaging portion being mechanically coupled to each other in a configuration in which the first plane and the second plane define any angle between 0° and 90°.

6. The device of claim 1, the first substantially circular engaging portion and the second substantially circular engaging portion being formed as an integral unitary structure.

7. The device of claim 1, the first substantially circular engaging portion and the second substantially circular engaging portion being formed as separate units configured to be mechanically coupled to each other.

8. The device of claim 1, further comprising a connecting portion interposed between the first substantially circular engaging portion and the second substantially circular engaging portion to mechanically couple the first substantially circular engaging portion and the second substantially circular engaging portion at a specified distance from each other.

9. The device of claim 8, the first substantially circular engaging portion, the second substantially circular engaging portion and the connecting portion being formed as an integral unitary structure.

10. The device of claim 8, the first substantially circular engaging portion and the second substantially circular engaging portion being formed as separate units configured to be mechanically coupled to each other via the connecting portion.

11. The device of claim 1, the second substantially circular engaging portion being formed in a substantially crescent shape with an upper opening to accommodate the blood tube holder.

12. The device of claim 11, a lower circular opening in the second substantially circular engaging portion having a diameter in a range of 22 mm.

13. The device of claim 11, the second substantially circular engaging portion being formed to have at least one protrusion that is configured to facilitate spreading the upper opening to accommodate the blood tube holder.

14. The device of claim 1, the second substantially circular engaging portion further comprising additional body structures extending in an axial direction from a main body structure of the second substantially circular engaging portion to support an additional axial length of the at least one of the blood tube holder or the another substantially tubular shaped component engaged by the second substantially circular engaging portion in use.

15. The device of claim 14, the additional body structures extending in the axial direction from the main body structure of the second substantially circular engaging portion by a distance of approximately 15 mm.

16. The device of claim 1, individual component structures of at least one of the first substantially circular engaging portion and the second substantially circular engaging portion having a principal cross-sectional dimension in a range of 5 mm.

17. The device of claim 1, at least one of the first substantially circular engaging portion and the second substantially circular engaging portion being formed of a nonporous material.

18. The device of claim 17, the nonporous material being one of a plastic, a polymer, and a metal material.

19. The device of claim 1, at least one of the first substantially circular engaging portion of the second substantially circular engaging portion being formed of a material including an antimicrobial substance.

20. A method for drawing blood from a patient, comprising:
providing a device to facilitate the blood draw, the device comprising:
a first substantially circular engaging portion that is configured to engage at least one of a thumb or a finger of a user, and
a second substantially circular engaging portion that is configured to engage a blood tube holder,
the first substantially circular engaging portion and the second substantially circular engaging portion being mechanically coupled to each other;
engaging the blood tube holder with the second substantially circular engaging portion of the device;
connecting one of a needle and a needle holder to the blood tube holder via an intermediate blood transmission tube;
engaging a thumb or finger of the user with the first substantially circular engaging portion to secure the device to a hand of the user;
piercing skin of the patient with the needle and engaging a vein in the patient with the needle;
inserting a blood draw tube into the device-engaged blood tube holder to facilitate the blood draw;
extracting the blood draw tube from the device-engaged blood tube holder when the blood draw tube is full;
withdrawing the needle from the patient when the blood draw evolution is complete;
dissembling and disposing of multiple blood draw components and the device.
